(19) United States
(12) Patent Application Publication
(10) Pub. No.: US 2008/0243187 A1
(43) Pub. Date: Oct. 2, 2008

(54) Vertebral body fixation apparatus

(75) Inventor: Dimitri Protopsaltis, Memphis, TN (US)

Correspondence Address: Medtronic Spinal and Biologics;Attn: Noreen Johnson - IP Legal Department, 2600 Sofamor Danek Drive, Memphis, TN, 38132, US

(73) Assignee: Warsaw Orthopedic, Inc.

(21) Appl. No.: 11/701,100

(22) Filed: Feb. 1, 2007

(51) Int. Cl. A61B 17/70; A61B 17/58

(52) U.S. Cl. 606/250; 606/246; 606/247

(57) ABSTRACT

A cage device is disclosed that includes, in one embodiment, a body and a pair of pedicle tabs attached thereto and potentially for connecting other orthopedic devices, such as anchors, screws, or rods, to the vertebral body replacement and potentially other vertebrae. The body also has a central opening for receiving orthopedic devices and/or osteogenic material.

The present disclosure relates to devices used in orthopedic surgical procedures. Specifically, the present disclosure relates to a device that can be used to abut a portion of a vertebra or as a vertebral body replacement, and is connectable to other orthopedic devices, such as rod(s) and anchor(s), and potentially other vertebrae.

Several techniques and systems have been developed for correcting and stabilizing damage or malformation of bones, especially the spine. Among them are systems that connect one or more elongated members, such as a rod or bar, to several vertebrae over one or more vertebral motion segments. Such elongated members can correct spinal curvature, provide support for injured or diseased tissue, or otherwise provide therapy. In many cases, such elongated members are connected to vertebrae by way of anchor members like screws (e.g. pedicle screws), hooks or other devices. For example, screws can be inserted directly into vertebral bone to provide a solid anchorage in the bone, and an elongated member can be connected either directly to the screws, or indirectly to the screws via connectors of various types.

In some cases, however, the available bone or other tissues is relatively small for screws or other anchors, or screws to be used with such bone may be small and difficult to handle. In the cervical region of the spine, for example, vertebrae and associated tissue are typically smaller as compared to vertebrae, disks and other tissues in other regions of the spine. The cervical vertebrae and disks may be difficult for surgeons to effectively and safely operate on while reducing the potential for damage to bone, cartilage, nerve or other tissue. Using screws, hooks or other anchors with such vertebrae, or in other regions of the spine in which the spinal cord or other sensitive tissue may be proximate to the anchorage site, requires particular devices as well as abundant care on the part of the surgeon.

### BRIEF DESCRIPTION OF THE DRAWINGS

FIG. 1 is a perspective view of one embodiment of a cage device.

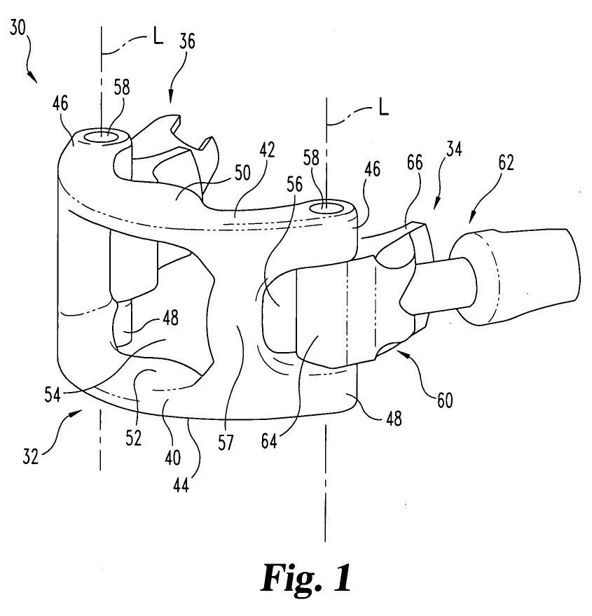

FIG. 2 is a further perspective view of the embodiment depicted in FIG. 1.

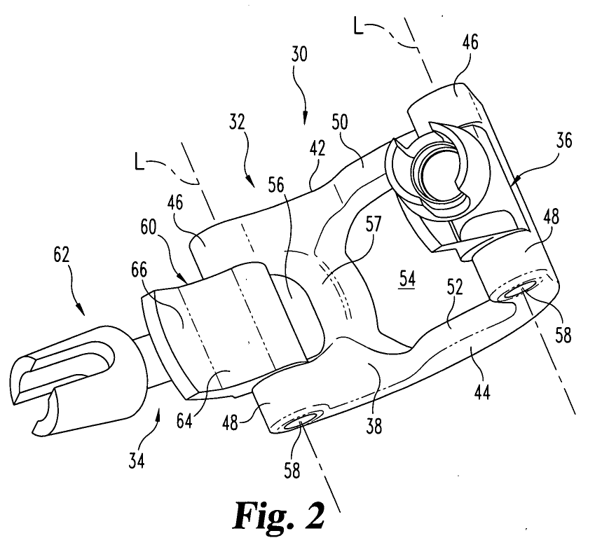

FIG. 3 is a further perspective view of the embodiment depicted in FIG. 1.

FIG. 4 is an exploded view of the embodiment depicted in FIG. 1.

FIG. 5 is a perspective view of a portion of the embodiment depicted in FIG. 1.

FIG. 6 is a cross-sectional view of the embodiment depicted in FIG. 5, taken along the lines VI-VI in FIG. 5 and viewed in the direction of the arrows, with additional structure not in cross-section.

FIG. 7 is an expanded view of another embodiment of a cage device.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated devices, and such further applications of the principles of the disclosure as illustrated therein, being contemplated as would normally occur to one skilled in the art to which the disclosure relates.

Referring generally to the figures, there are shown embodiments of a cage device 30. In the illustrated embodiment, device 30 includes a main cage body 32, a first tab or anchor 34, and a second tab or anchor 36. As discussed further below, device 30 can be connected to bone or other tissue, for example around a vertebra or between vertebrae, and can be used in connection with a variety of orthopedic devices, such as orthopedic plates, connectors, rods, fusion cages, disk prostheses, or other types of instrumentation or implants. Device 30 may also be used in connection with osteogenic compositions, if desired.

In the illustrated embodiment, body 32 includes an interior or front side 38 and an opposite exterior or rear side 40. Body 32 includes a relatively superior surface 42 and an opposite relatively inferior surface 44, that in a particular embodiment are generally perpendicular to at least part of interior side 38 and exterior side 40. Body 32 may further include one or more relatively superior side arms 46, and in the illustrated embodiment two such arms 46 are provided, each of which having a surface that is substantially coplanar to surface 42. Body 32 may also include one or more relatively inferior side arms 48, and in the illustrated embodiment two such arms 48 are provided, each of which having a surface that is substantially coplanar to surface 44. Arms 48 are generally opposite arms 46. In one embodiment, body 32 has a substantially concave shape sized and configured so that interior side 38 substantially abuts or fits adjacent a surface of a vertebra, e.g. a cervical vertebra, as described in more detail below. Such an embodiment of body 32 may be thought of as generally C-shaped, with each pair of arms 46, 48 forming sides or ends of the C-shape.

Interior surface 38 includes projections 50 and 52 in the illustrated embodiment. Projection 50 is located between arms 46, and in a particular embodiment is centered between arms 46 and has a surface that is coplanar with surface 42. Projection 52 is located between arms 48, and in a particular embodiment is centered between arms 48 and has a surface that is coplanar with surface 44. Projections 50 and 52 may be thought of as extending inward from the remainder of body 32, or generally in the same direction as arms 46 and 48, and generally toward a vertebra or intervertebral disk space to or in which device 30 is connected. Multiple such projections may be placed on device 30, e.g. two or three such projections may be generally evenly spaced between arms 46 or 48. As shown, projections 50 and 52 may each be substantially convex and rounded, and may form part of a circle or cylinder. In some embodiments, projection 50 can engage a top end plate of a vertebral body, and projection 52 can engage a bottom end plate of the same vertebral body, thus in essence gripping the vertebra on top and bottom. In other embodiments, projection 50 may engage a bottom end plate of a superior vertebra, and projection 52 may engage a top end plate of an adjacent inferior vertebra, thus separating the vertebrae and acting as an intervertebral body replacement. In these embodiments, one or both projections 50 and 52 may have rounded or slanted top and/or bottom surfaces to make it easier to place device 30 adjacent to or abutting vertebral endplates.

Body 32 defines a central opening 54 in the illustrated embodiment, which is located between projections 50 and 52 and extends between interior side 38 and exterior side 40. As shown, opening 54 is substantially octagonal in shape so as to provide sturdiness and stability to device 30, although it will be seen that opening 54 could be substantially triangular, square, circular, or otherwise configured. Opening 54 can be sized, for example, to receive a variety of orthopedic instrumentation, or may be packed with osteosynthetic compositions, as further described below. One or more side openings 56 may also be provided in body 32. In the illustrated embodiment, two openings 56 are provided, each located between an arm 46 and an arm 48, and each being substantially C-shaped. Between openings 54 and 56 is found a post or bridge portion 57, which may be thought of as separating and supporting the superior and inferior portions of body 32. As is further discussed below, side openings 56 are sized and configured to receive one of tabs 34 and 36.

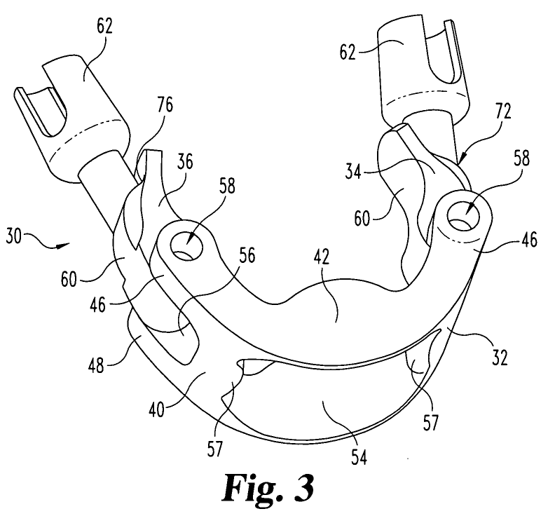

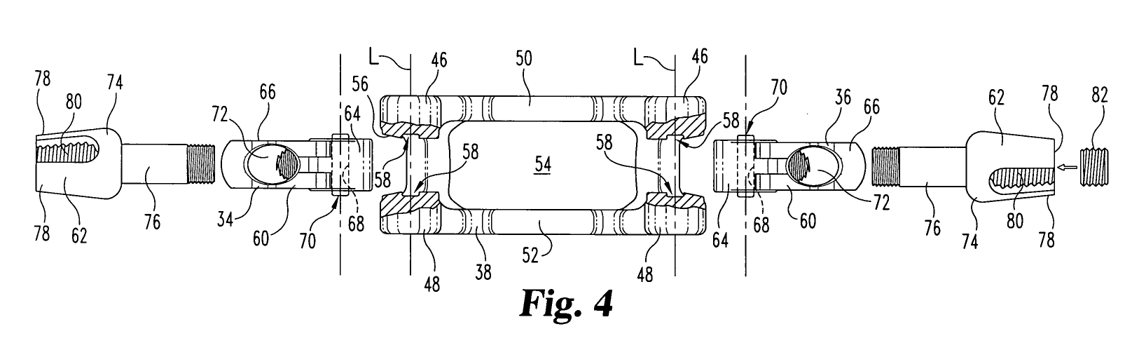

Each of arms 46 and 48 of the illustrated embodiment include a hole or pivot point 58. A given hole 58 may be open, extending all the way through its respective arm (FIG. 3), or closed, extending only part of the way through its respective arm (FIG. 4). An axis L extends through the centers of holes 58 of adjacent arms 46 and 48 in the illustrated embodiment. As further discussed below, tabs 34 and 36 are each pivotally attached to an arm 46 and an arm 48 by way of an axle, pin, boss(es) or other structures inserted at least partially into holes 58 of the given arm 46 and/or 48.

As shown in FIG. 4, the illustrated embodiment of tab 34 has connecting portion 60 for connecting to body 32, and a rod anchoring portion 62 for connecting to and/or holding an elongated member such as a spinal rod. Tab 36 is substantially identical to tab 34 in this embodiment, and therefore the following discussion with respect to tab 34 is equally applicable to tab 36. Connecting portion 60 includes a base 64 that is sized and dimensioned to fit in an opening 56, between arms 46 and 48 of body 32, and a tab portion 66 that is substantially flat or curved and extends away from base 64. Base 64 includes an aperture 68 through which a pin 70 extends, in this particular embodiment. Pin 70 extends from aperture 68 on at least one side (on both sides in the illustrated embodiment), and can be inserted into one or both of holes 58 and between arms 46 and 48. In this way, tab 34 can be rotated within arms 46 and 48 about axis L. Tab portion 66 provides a surface or contact point to enable one to pivot tab 34. Tab 34 also defines an opening 72 sized and configured to receive anchoring portion 62 or another connector, rod, instrument or other device. In the illustrated embodiment, opening 72 is at least partially threaded, but in other embodiments may be substantially smooth, or include grooves or other features for holding anchoring portion 62.

The illustrated embodiment of anchoring portion 62 has a receiving portion 74 and a shaft portion 76. Receiving portion 74 has two branches 78 defining a channel 80 between them for accommodating at least part of a rod or other elongated member R. A locking member 82 is provided to connect with receiving portion 74 so as to hold rod R in channel 80. As one example, locking member 82 may be a set screw, and branches 78 may be internally threaded to accommodate the set screw. Shaft portion 76 of anchoring portion 62 is sized for insertion into opening 72 of tab 34. In the illustrated embodiment, shaft 76 is threaded so that it can be threaded into opening 72. In that embodiment, anchoring portion 62 is inhibited from slipping out of opening 72, yet can be rotated so as to orient channel 80 in a desired direction. In other embodiments, shaft 76 may have other features, such as bosses or detents, that maintain it within opening 72, or other additional structure (e.g. a clamp) could be provided to hold shaft 76 in hole 72. In yet other embodiments, anchoring portion 62 may be made integrally with connecting portion 60, or the two parts may be fixed together as with adhesives or by welding.

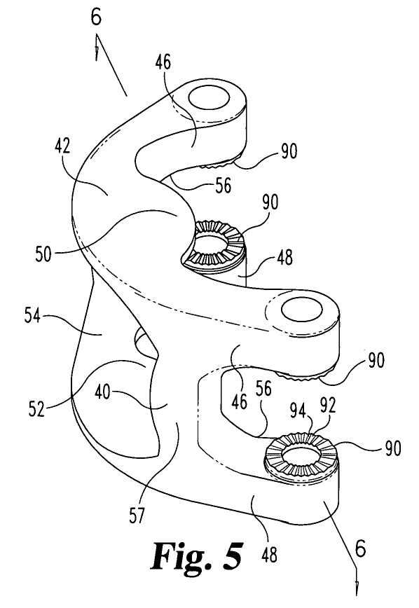

In one particular embodiment, as shown in FIG. 5, each of superior arms 46 and each of inferior arms 48 include an indexing feature 90 for adjusting the position of tabs 34 or 36. In this embodiment, indexing feature 90 includes a plurality of ridges or splines 92 and a plurality of grooves 94 arranged in a substantially circular pattern, each ridge 92 separated from another ridge 92 by a groove 94. Ridges 92 may be substantially trapezoidal, triangular, or otherwise shaped. In one form, indexing feature 90 includes a locking feature for locking a desired position of tab 34 or of tab 36. Corresponding with indexing feature 90, one or both of tabs 34 and 36 may include a plurality of ridges or splines 96 and a plurality of grooves 98 configured such that ridges 96 fit with grooves 94 of indexing feature 90, and grooves 98 assemble with ridges 92 of indexing feature 90. Additionally, ridges 96 and grooves 98 of tabs 34 and/or 36 may also be configured to traverse ridges 92 and grooves 94 of indexing feature 90. For example, tab 34 may rotate about axis L while ridges 96 traverse ridges 92, and tab 34 may be positioned at any of a number of discrete positions relative to body 32.

Device 30 may be assembled (as described above) prior to use in a surgical procedure. In the description below, a method of using device 30 will be described in the context of spinal surgery. It will be seen that device 30 could be used in a number of orthopedic situations, and the description in the spinal context is not intended to be limiting.

In using the illustrated embodiment of device 30, a surgeon obtains access to the vertebra(e) to be worked on, via an open or other approach, with movement or resection of adjacent tissues as the surgeon may deem necessary. For example, portions or entireties of vertebra(e) or intervertebral disk(s) may be removed. Once the surgical site is suitably prepared, device 30 is placed. In a procedure in which device 30 is used as a surrogate for attachment of anchors to a vertebra, interior side 38 of device 30 is positioned against a vertebral surface. In one particular embodiment, interior surface 38 may be placed against an anterior portion of a vertebra. Projection 50, if provided, may be positioned to contact and engage the top endplate of the vertebra while projection 52, if provided, may be positioned to contact and engage the bottom endplate of the same vertebra. If necessary, the surgeon can create a small channel or otherwise remove some bone tissue so that projections 50 and/or 52 can fit closely but not uncomfortably with the vertebra. Arms 46 and 48 wrap partially around the vertebra, and may be manipulated by the surgeon in some embodiments, as by bending, to fit relatively closely or snugly against the vertebra. Body 32 may be affirmatively attached to the vertebral surface by adhesive or other substance or method. Central opening 54 can be packed with osteogenic materials such as BMP, LIM mineralization protein, allograft, autograft, or other substances, so as to create a bony connection to or capsule around at least part of body 32.

In a medical procedure where part or all of a vertebra or intervertebral disk is replaced, the surgeon can insert device 30 between an upper vertebra and a lower vertebra. Surface 42 may contact and engage the bottom endplate of one vertebra while surface 44 may contact and engage the top endplate of another vertebra. In this form, surfaces 42 and 44 and arms 46 and 48 extend partially around a circumference or other portion of the adjacent vertebrae, separating and supporting them. Central opening 54 can be packed with osteogenic materials, such as those described above, so that a fusion between the vertebrae, or among the vertebrae and body 32 may be more easily achieved. Other substances or devices for creating a secure connection between the vertebrae and body 32 may be used.

If not already positioned as desired, tabs 34 and 36 may be rotated into the desired position. Thus, prior to insertion of device 30, tabs 34 and 36 may be “wide,” i.e. pointing outward from the respective arms 46 and 48 to which they are connected. Once interior surface 38 is against an anterior portion of a vertebra, or surfaces 42 and 44 are between vertebrae, tabs 34 and 36 may be rotated so that their anchoring portions 62 extend to a postero-lateral position with respect to the vertebra, e.g. substantially between the transverse and spinious processes. In certain embodiments, tabs 34 and 36 can be positioned against bone or other tissue, so that device 30 is lightly or strongly clamped to the vertebra.

Once device 30 is attached to a vertebra or placed as discussed above, a connector, anchor, rod or other elongated member, or other orthopedic device can be coupled with device 30. Using a spinal rod R and tab 34 as an example, the rod may be inserted into channel 80 of anchoring portion 62, and held or locked therein with locking member 82. Tab 34 may be rotated with respect to body 32 in order to make insertion of rod R in to channel 80 easier, and/or as a means of repositioning rod R. In such a case, rod R may be inserted into channel 80, locking member 82 may be loosely connected to anchoring portion 62, tab 34 may be rotated to a desired position, and locking member 82 may be firmly locked against rod R and/or anchoring portion 62 so that motion of rod R with respect to anchoring member 62 is limited or prevented. It will be observed that the illustrated embodiment of device 30 enables connection to at least one rod at each tab 34, 36. Thus, a similar or identical connection method can be practiced with respect to a second rod and tab 36, resulting in a dual rod implant construct.

In other embodiments, other types of connections between implant parts and device 30 can be made. For example, other types of connectors or implants may be inserted and/or locked into opening 72 of tabs 34 and/or 36. A rod or other elongated member may also be inserted directly into opening 72, particularly if such elongated member has a bend adjacent an end, so that that end portion can be inserted and/or locked into opening 72, while the remainder of the elongated member extends along a portion of the spine.

Other orthopedic procedures can be performed before, during or after connection of one or more rods to device 30. For example, compression, rotation or distraction of vertebrae can occur prior to attaching device 30 to vertebra(e) or when rods are loosely connected to it, or at other points during the surgery. As another example, a fusion cage, prosthesis or other type of intervertebral implant may be placed at or near the location of device 30, so as to provide additional support or correct other problems. Once the surgeon has locked rod(s) with respect to device 30, and performed any other procedures deemed necessary or prudent, the surgery can be ended and the incision(s) closed.

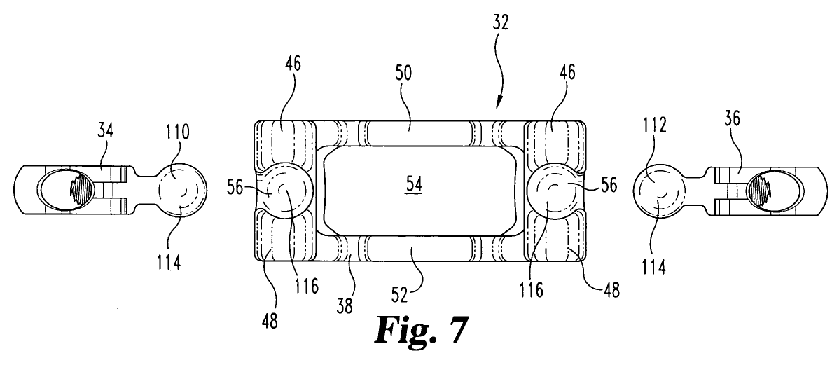

Variations in the structure of device 30 are contemplated. For example, in another embodiment shown in FIG. 7, tab 34′ includes an anchor member 110 and tab 36′ includes an anchor member 112. Anchor member 110 is configured to fit within a side opening 56, which is particularly configured to receive a part of anchor member 110. In this embodiment, anchor member 110 includes a rounded extension 114 and side opening 56 having a substantially round cavity 116. Extension 114 and cavity 116 may both be at least partly spherical in a particular embodiment. Extension 114 and cavity 116 thus form a ball-and-socket arrangement such that anchor member 110 can rotate within side opening 56 with at least two degrees of freedom. Likewise, the other side opening 56 and anchor member 112 are similarly fashioned in this particular embodiment, so that anchor member 112 and the other side opening 56 also form a ball-and-socket arrangement such that anchor member 110 can rotate within side opening 56 with at least two degrees of freedom. Other aspects of tabs 34′ and 36′ are substantially the same as those of tabs 34 and 36, described above, and other aspects of body 32 are substantially the same as those described above.

In other embodiments, tab 34 and/or tab 36 may have anchoring portions that are multi-axial or otherwise pivotable. For example, an anchoring portion similar to anchoring portion 62 but having a shaft separate from the receiving portion so that the receiving portion can rotate or pivot multi-axially with respect to the shaft can be provided. Examples of such structures are shown and described in commonly-owned U.S. Pat. No. 6,280,442, the disclosure of which is hereby incorporated by reference.

It is further contemplated that, instead of a pin or axle 70 extending through tabs 34 and/or 36, one or both of their bases 64 could be substantially solid, with a nub or protrusion extending from one or both ends and fitting into hole(s) 58. FIG. 4 shows a relatively small amount of pin 70 extending from each tab 34 and 36. Such nubs likewise could extend from base 64 of a given tab. Such nub(s) will allow rotation of tabs 34 and/or 36 with respect to body 32, as previously discussed.

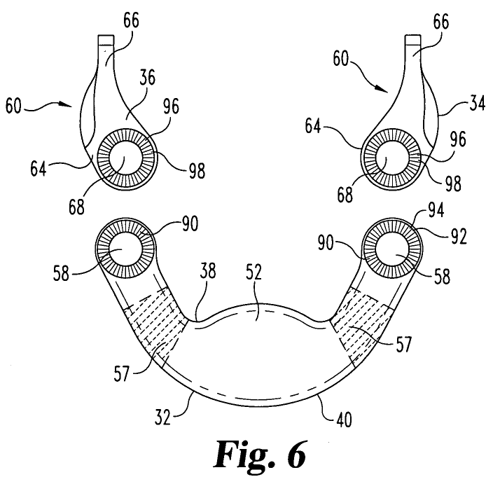

Still other embodiments of device 30 are contemplated as being within the scope of the present disclosure. In one embodiment, body 32 has a shape similar to the periphery of a vertebra such that interior side 38 fits snugly against the vertebra. For example, projections 50 and 52 can be of different shapes, or may be similarly shaped as indicated above. In one form, projections 50 and/or 52 may be rectangular in shape. In another embodiment, central opening 54 can be another shape such as, but not limited to, rectangular, circular, polygonal, or triangular. In other embodiments, side openings 56 can be shaped independently of each other or similar to each other. As shown in FIGS. 4, 5, 6, and 7, tab 34 and/or tab 36 can each be attached to one arm 46 and one arm 48 in a variety of ways. Tab 34 and tab 36 can be attached to body 32 in ways that are alike, or one can be connected in one way and the other in a different way. For example, tab 34 could be attached as shown in FIG. 4 while tab 36 could be attached as shown in FIG. 5. Further, structure other than anchoring portion 62 could be connected to tabs 34 and/or 36 via aperture 72. Thus, rods could be inserted into aperture 72 and connected to device 30, or connectors, clamps or other devices could be placed in aperture 72 and connected to rods or other structure.

Device 30 may be made of any biocompatible material. Biocompatible metals include titanium, stainless steel, titanium alloys, nickel-titanium alloys, nitinol, and chrome alloy, to name a few.

Biocompatible plastics include polyurethane, polyester, polyether, polyalkene, polyethylene, polyamide, polyvinyl fluoride, polyether ether ketone (PEEK), or polytetrafluoroethylene (PTFE). In certain embodiments, body 32, tab 34, and tab 36 are made of the same material, and in particular embodiments body 32, tab 34, and tab 36 are made of PEEK or other flexible material. In that particular embodiment, central opening 54 can expand or flex as a mechanical force is applied to body 32. It has been found that where body 32, tab 34, and tab 36 made of PEEK or other radiolucent material, x-ray and MRI artifacts are minimized. In one example, by minimizing x-rays and MRI artifacts such as at surfaces 42 and 44, a surgeon can potentially extract valuable information concerning the spinal column and vertebral arteries when device 30 is attached to a vertebra. It will be seen that body 32, tab 34, and tab 36 could be made of different materials. For example, body 32 may be made of a biocompatible plastic, and tabs 34 and 36 may be made of a biocompatible metal.

While the disclosure has been illustrated and described in detail in the drawings and foregoing description, the same is to be considered as illustrative and not restrictive in character, it being understood that only the preferred embodiment has been shown and described and that all changes and modifications that come within the spirit of the disclosure are desired to be protected.

What is claimed is:

1. An orthopedic medical apparatus comprising:
a body having a concave surface, a convex surface and first and second side portions;
a first tab movably connected to said first side portion, said first tab including a receiving portion for receiving an elongated member; and
a second tab movably connected to said second side portion, said second tab including a receiving portion for receiving an elongated member.

2. The apparatus of claim 1, wherein at least one of said tabs is pivotable with respect to its respective side portion.

3. The apparatus of claim 1, wherein at least one of said tabs is multi-axially rotatable with respect to its respective side portion.

4. The apparatus of claim 1, wherein at least one of said receiving portions is substantially U-shaped and internally threaded.

5. The apparatus of claim 4, further comprising a set screw threadable into said at least one of said receiving portions.

6. The apparatus of claim 1, wherein said receiving portion of said first tab is fixed with respect to the rest of said first tab.

7. The apparatus of claim 2, wherein said receiving portion of said second tab is fixed with respect to the rest of said second tab.

8. The apparatus of claim 1, wherein said body has an opening between said side portions.

9. The apparatus of claim 8, wherein said opening is sized and configured to permit insertion of at least one osteogenic substance.

10. The apparatus of claim 1, wherein said first side portion includes first and second arms, and said First tab includes a base portion connected to said arms.

11. The apparatus of claim 10, wherein said first and second arms each have a respective aperture, and said base portion includes a hole therethrough, and further comprising a pin inserted through said hole of said base portion and into said apertures of said arms.

12. The apparatus of claim 10, wherein said first and second arms each have a respective aperture, and said base portion includes a pair of opposed projections, each said projection being inserted at least partially into a respective one of said apertures of said arms.

13. The apparatus of claim 10, wherein at least one of said arms has a surface that includes radial splines, and said base portion includes a surface having compatible splines, said splined surface of said base portion engaging said splined surface of said arm.

14. The apparatus of claim 1, wherein at least a portion of said concave surface is configured approximately in the shape of at least a portion of an anterior surface of a vertebra.

15. The apparatus of claim 1, wherein said body includes a substantially superior surface and a substantially inferior surface, and further comprising first and second extensions, said first extension extending from said concave surface and being adjacent said substantially superior surface, and said second extension extending from said concave surface and being adjacent said substantially inferior surface.

16. The apparatus of claim 15, wherein said first extension has a portion that is coplanar with said substantially superior surface, and said second extension has a portion that is coplanar with said substantially inferior surface.

17. An orthopedic medical apparatus comprising:
a C-shaped body having two separated side portions; and
a tab pivotably connected to one of said side portions, said tab adapted to receive an orthopedic implant.

18. The apparatus of claim 17, wherein said tab includes a hole, and further comprising an orthopedic implant inserted into said hole.

19. The apparatus of claim 18, wherein said orthopedic implant is from the group consisting of anchor members having a shaft portion, connector devices, and orthopedic rods.

21. The apparatus of claim 17, wherein said tab includes a substantially U-shaped receiving portion for receiving an orthopedic implant.

22. The apparatus of claim 17, further comprising a second tab pivotably connected to the other of said side portions, said tab adapted to receive an orthopedic implant.

23. The apparatus of claim 17, wherein said body is plastically deformable.

24. A method comprising:
providing an orthopedic medical apparatus having a C-shaped body with two separated side portions, a first tab pivotably connected to a first of said side portions, and a second tab pivotably connected to a second of said side portions, at least one of said tabs being adapted to receive an elongated member;
placing said apparatus adjacent at least one vertebra;
pivoting at least one of said tabs into a desired position; and
connecting an elongated member to said tab adapted to receive the elongated member.

25. The method of claim 24, further comprising locking said elongated member with respect to said tab.

26. The method of claim 24, wherein both of said tabs are adapted to receive an elongated member, and wherein said connecting step connects a first elongated member to a first said tab, and further comprising connecting a second elongated member to a second said tab.

27. The method of claim 24, further comprising connecting said elongated member to at least one other orthopedic implant.

28. The method of claim 24, further comprising bending said body so that said body closely approximates or grips the vertebra.

29. The method of claim 24, further comprising placing osteogenic material in or adjacent to said body.

30. The method of claim 24, wherein said body has a substantially concave surface, and said placing step includes locating at least a portion of said concave surface adjacent at least a portion of an anterior surface of the vertebra.

31. The method of claim 24, wherein said body has a substantially superior surface and a substantially inferior surface, and said placing step includes locating said substantially superior surface adjacent an endplate of a relatively superior vertebra and locating said substantially inferior surface adjacent an endplate of a relatively inferior vertebra.

32. The method of claim 31, further comprising inserting an orthopedic implant between said relatively superior vertebra and said relatively inferior vertebra and substantially between said side portions of said body.
